(19) United States
(12) Patent Application Publication
(10) Pub. No.: US 2005/0166272 A1
(43) Pub. Date: Jul. 28, 2005

(54) Enhancement of RNAi activity through mutation

(75) Inventors: Kazunari Taira, Tsukuba (JP); Makoto Miyagishi, Tsukuba (JP); Sahohime Matsumoto, Tokyo (JP)

Correspondence Address: FOLEY AND LARDNER;SUITE 500, 3000 K STREET NW, WASHINGTON, DC, 20007, US

(73) Assignee: National Institute of Advanced Industrial Science and Technology

(21) Appl. No.: 11/006,804

(22) Filed: Dec. 8, 2004

Related U.S. Application Data
(60) Provisional application No. 60/527,766, filed on Dec. 9, 2003.

(51) Int. Cl. A01K 67/00; C07H 21/02; C12N 5/04; A61K 48/00; A01H 1/00; C12N 15/82

(52) U.S. Cl. 800/8; 800/286; 514/44; 435/325; 435/419; 536/23.1

(57) ABSTRACT

A double-stranded RNA comprising an antisense RNA and a sense RNA with specific mutations are disclosed as having enhanced RNAi activities. The double stranded RNA has at least one substitution or insertion mutation introduced at a first, second or third nucleotide position from the 5′ end of the antisense RNA or at 17-19 position as counted from the 3′ end of the antisense sequence excluding an overhang, and the antisense RNA is complementary to a region of an mRNA of a target gene except the at least one substitution or insertion mutation. Related single-strand RNAs, vectors, methods, cells and compositions are disclosed.

### BACKGROUND OF THE INVENTION

The present invention generally relates to the enhancement of RNAi activity and more specifically to a double-stranded RNA, vector, and method for effectively inhibiting expression of the mRNA of a target gene.

RNA interference (RNAi) is a phenomenon in which double-stranded RNAs (dsRNA) cleave complementary target mRNAs. The phenomenon was first reported by Fire et al. (Fire et al., 1998) in Caenorhabditis elegans, and subsequently observed in mammalian cells as well (Caplen et al., 2001; Elbashir et al., 2001a; Elbashir et al., 2001b). When a dsRNA is introduced into a cell, it is cleaved by an RNase III family member called Dicer into 21 to 23 nucleotide (nt) RNAs with 2 or 3 nt overhangs at 3′ ends (small interfering RNAs; siRNAs) (Bernstein et al., 2001; Elbashir et al., 2001b). Subsequently, the siRNAs combine with RNA-induced silencing complex (RISC), and the RISC catalyzes sequence-specific cleavage of the targeted mRNA by using the siRNA as a guide sequence (Bernstein et al., 2001; Hammond et al., 2001). The siRNA has attracted a great deal of attention not only from its mechanistic point of view but also as useful new tools for numerous gene-silencing experiments.

However, the technique of introducing synthetic siRNAs into cells has some limitations, including the short-term persistence of gene-silencing activity, the variability of transfection efficiency and the cost of synthesizing siRNAs. Several groups, including the present inventors, have circumvented these problems, by developing vector-mediated RNAi expression system (Brummelkamp et al., 2002; Lee et al., 2002; Miyagishi and Taira, 2002; Paddison et al., 2002; Paul et al., 2002; Sui et al., 2002; Yu et al., 2002). The plasmid-based siRNA expression approach allows longer lasting RNAi, especially when stable transfection or transduction of cells can be achieved. The siRNA expression vector systems could increase the possibilities of therapeutic applications of RNAi (Lee et al., 2002; McCaffrey et al., 2003; Novina et al., 2002; Song et al., 2003). Despite the potency of RNAi, there are still many unsolved questions about the design of siRNA. Although it was thought that siRNAs with perfect complementarity to the target are most effective at diverting cleavage, recent studies indicated that functional siRNAs are not always completely matched with the target mRNA (Boutla et al., 2001; Amarzguioui et al., 2003; Chi et al., 2003; Holen et al., 2003; Jackson et al., 2003; Semizarov et al., 2003).

### SUMMARY OF THE INVENTION

For the present invention, the sequence specificity of RNAi has been systematically investigated. To understand what types of mutations and in which positions still preserve the activity of RNAi, a series of mutated siRNA expression vectors, which include transversion, deletion and insertion mutations in various positions of 21 nt strands and strands of other lengths have been constructed. Importantly, it has been found that possession of A or U at the 5′ end can always enhance RNAi activity.

The present invention provides a double-stranded RNA comprising an antisense RNA and a sense RNA, wherein the double stranded RNA has at least one substitution or insertion mutation introduced at a first, second or third nucleotide position from the 5′ end of the antisense RNA and the antisense RNA is complementary to a region of an mRNA of a target gene except the at least one substitution or insertion mutation.

The present invention also provides a double-stranded RNA comprising an antisense RNA and a sense RNA, wherein the double stranded RNA has at least one substitution or insertion mutation introduced at any of nucleotide positions 17 to 19 as counted from the 3′ end of the antisense sequence excluding an overhang and the antisense RNA is complementary to a region of an mRNA of a target gene except the at least one substitution or insertion mutation.

Further, the present invention provides related single-strand RNAs, vectors, methods, cells and compositions.

The substitution mutation, as used herein, means substitution of one or more of nucleotides in a RNA sequence and is meant to cover a transverse mutation. The transverse mutation is defined as the interchange of pairing nucleotides at one position in a double-stranded RNA sequence. As used in the present specification, transverse mutation is not limited to mutations between pyrimidines and purines and may include what is often called transitions which are mutations from a pyrimidine to a purine and vice versa. Thus, the substitution mutation mentioned above may be a transverse mutation. The insertion mutation may be defined as the insertion of a nucleotide at one position in an antisense RNA sequence. In the present specification, the insertion of a nucleotide at position x is meant to occur on the 5′ side of a nucleotide located at position x as viewed with reference to the 3′-5′ direction of the antisense RNA.

The double-stranded RNA of the present invention may have a length of 19 nt and the at least one substitution or insertion mutation may be at any of nucleotide positions 17 to 19 as counted from the 3′ end of the antisense sequence excluding an overhang while the mutation at the 5′ end of the antisense RNA is preferred. The mutations may involve either one nucleotide pair or one nucleotide or possibly either two nucleotide pairs or two nucleotides.

The length of the double-stranded RNA may be 15-100 nt, preferably 17-50 nt, and more preferably 19-30 nt. Also, the RNA may have an overhang of 2-4 nt length. Further, the double-stranded RNA can be transcribed using Pol III or Pol II promoter.

The present invention also provides a double-stranded RNA comprising an antisense RNA whose 5′end nucleotide is adenosine or uridine in place of guanosine complementary to cytidine present in a region of an mRNA of a target gene and which is complementary to the region of the mRNA of the target gene except at the 5′ end; and a sense RNA which is complementary to the region of the mRNA of the target gene. In such double-stranded RNA, cytidine in the sense RNA corresponding to the replaced adenosine or uridine is replaced by uridine or adenosine respectively so as to form a complementary pair at the 5′ end of the antisense RNA.

The present invention further provides a double-stranded RNA comprising an antisense RNA whose 5′end nucleotide is adenosine or uridine in place of cytidine complementary to guanosine present in a region of an mRNA of a target gene and which is complementary to the region of the mRNA of the target gene except at the 5′ end, and a sense RNA which is complementary to the region of the mRNA of the target gene. In such double-stranded RNA, guanosine in the sense RNA corresponding to the replaced adenosine or uridine is replaced by uridine or adenosine respectively so as to form a complementary pair at the 5′ end of the antisense RNA.

In fact, for the double-stranded RNA having a length of 19 nt, the substitution or insertion mutation can be at any position except nucleotide positions 13 to 16 as counted from the 3′ end of the antisense sequence excluding an overhang. Preferably, the substitution or insertion mutation can be at any of nucleotide positions 17-19. The substitution or insertion mutations may also be at any of nucleotide positions 1-4 as well.

The double-stranded RNA described above may also have a loop strand connecting the sense and antisense strands to have a hairpin or stem-loop configuration.

The double-stranded RNA may comprise an antisense RNA whose 5′end nucleotide is modified to be adenosine or uridine replacing guanosine corresponding to cytidine found in a region of the target gene mRNA sequence, and which is otherwise complimentary to the sequence found in the region, and a corresponding sense RNA.

The present invention also provides a vector for inhibiting expression of an mRNA of a target gene, comprising: an antisense DNA strand coding for an antisense RNA which has at least one mutation that would correspond to a substitution or insertion mutation at or near the 5′ end of a double-stranded RNA resulting therefrom and is otherwise complementary to the mRNA; and a promoter operatively linked to the sense DNA. The vector may be hairpin type in which the antisense DNA strand and a corresponding sense strand are positioned downstream from the promoter or a tandem type in which the antisense DNA strand and a corresponding sense strand are positioned downstream from each of two promoters. The hairpin type appears preferable to the tandem type due to the fact that the hairpin type produces more stable antisense RNAs in cells than sense RNAs. In the hairpin type vector, the promoter may preferably be followed by the sense strand and then the antisense strand. The vector may further comprise a terminator which is located downstream of the antisense DNA and capable of terminating the transcription of the antisense DNA.

The present invention further provides a method for inhibiting expression of an mRNA of a target gene, comprising the steps of: preparing a double-stranded RNA comprising an antisense RNA which has at least one transverse or insertion mutation near the 5′ or 3′ end of the antisense RNA and which is complementary to some region of an mRNA of a target gene except the at least one transverse or insertion mutation; and a sense RNA having a sense sequence complementary to the antisense RNA; and contacting the mRNA with the double-stranded RNA in an environment suitable for the antisense RNA of the double-strand RNA to hybridize with the mRNA.

The vectors of the present invention may further contain, as required, a selection marker that enable the selection of cells into which a vector has been introduced. Selection markers may include a drug-resistant marker such as genes resistant to neomycin, hygromycin or puromycin, markers that can select based on enzymatic activities such as galactosidase activity, and markers that use phosphorescence, such as GFP. Also, selection markers that uses surface antigens such as EFG receptors, B7-2 or CD4. Using such selection markers, cells into which a vector or an siRNA expression system can be identified, and therefore problems associated with generally low efficiency of exogenous introduction of siRNA fragments into cell can be alleviated.

The present invention also provides cells that contain the siRNA expression system discussed above. Such cells are preferably mammalian cells simply because the siRNA expression system of the present invention is aimed at the induction of RNAi in mammalian cell, which has been difficult. Also, the present invention is preferably applicable to plant cells that contain as a target a long-chain dsRNA. However, the cells of the present invention are not limited to mammal or plant cells, but also cover animal cells other than mammalian cells, yeast or fungi cells.

The above-mentioned siRNA expression system may be introduced into cells in any suitable manner. For example, standard methods such as the use of electroporation, calcium phosphate, lipofection, viruses, gene or particle gums, or polyethyleneglycol can be used.

Methods for selecting cells that have been transfected with the siRNA expression system include a variety of known methods such as the use of hybridization using a DNA sequence specific to the siRNA expression system as a probe or primer, or PCR. Also, selection markers mentioned above can be used as well.

Cells that have been transfected with the siRNA expression system are called knock-down cells in which the expression of a target gene is suppressed. Here, the concept of “knock-down cells” covers cells in which the expression of the target gene is completely or partially suppressed. Conventionally, such cells are prepared by delete or modify a target gene or its control region. Using the present invention, knock-down cells can be prepared by the transfection of relevant cell with the siRNA expression system and selection of transfected cells, without complicated direct manipulation of genes. The knock-down cells of the present invention can be used for the functional analysis of target genes or as disease-associated model cells by suppressing the expression of genes that cause certain diseases. The siRNA expression system may be introduced into generative cells so as to produce transformants, such as knock-down animals or plants or disease model animals or plants.

Target-gene nock-down animals can be prepared using any known methodologies. For example, an siRNA expression vector can be introduced into a fertilized egg obtained by mating F1 female and male animals. Peripheral DNA is obtained from the tail of a mouse resulting from the egg. Positive animals that have been transfected with the siRNA expression system are identified, for example, by a genomic Southern blot using a part of the expression vector as a probe. Posterior mice can be produced by backcrossing.

The siRNA expression system can also be used for plants. Particularly, conventionally RNAi is induced by directly introducing double-stranded RNAs into plant cell, but the dsRNAs tend to disappear and RNAi activities are lost while cells are passaged. Therefore, RNAi activities may be maintained by integrating the siRNA expression system of the present invention into chromosomes in plant cells. Likewise, stable transgenic plants may be created using cells transfected according to the present invention.

Furthermore, the present invention provides compositions containing the siRNA expression system of the present invention. Such compositions include the vector or siRNA expression system of the present invention and a suitable vehicle and can be used for a variety of situations including experiments or medical testing or research purposes.

### BRIEF DESCRIPTION OF THE DRAWINGS

FIG. 1 generally shows the way the systematic analysis of various mutations in siRNA was conducted. FIG. 1A is a schematic representation of the precursor hairpin RNA, which is expressed from hairpin type siRNA-expression vector, and the processed siRNA The semicircle represents the 11 nt sequence of the loop strand. The lines indicate cleavage positions by a Dicer as the bottom schema designated. The each nucleotide is numbered 1 to 19 from 3′ end of the antisense strand except the 3′ overhang of 2 nt length. FIG. 1B shows the suppressive activity for a series of transversion mutations. The mutated bases are marked with squares. The term “nc” means negative control and “tra4,” for example, means transverse mutation at position 4. FIG. 1C shows the suppressive activity of deletion mutations which are prepared by cutting off one base from the antisense strand. The term “del4,” for example, means deletion mutation at position 4. The deleted base is shown with a star. FIG. 1D shows the suppressive activity of insertion mutations which are prepared by one base pair placed in the antisense strand. The inserted base is shown with arrows, and can be seen in the drawing, when insertion is noted to occur at position N (insN), a base is actually inserted between positions N and N+1 with respect to the from-3′-to-5′ direction of the antisense RNA or the right hand side of the base at position N. The term “ins4,” for example, means insertion between positions 4 and 5, and “Ins 2mer” means the insertion of a dimer UU at position 4

FIG. 2 generally shows the hairpin and tandem type vectors. FIGS. 1A and 1B show the Northern analysis of tandem type and hairpin type siRNA-expression vector with antisense probe (FIG. 2A) and sense probe (FIG. 2B). HeLa S3 cells were transfected with a tandem type or hairpin type siRNA-expression vector. After the sense analysis, the membrane was boiled off the probe and re-hybridized with the sense probe to detect the antisense strand. FIG. 2C shows the flow chart of the tandem type and hairpin type siRNA expression vector and processing pathways toward siRNA.

FIG. 3 generally shows the enhanced effects of 5′ end mutations of siRNA on suppressive activities of luciferases. FIG. 3A shows the dependence of 5′ end base of antisense strand on the suppressive activity. The mismatches are shown with diagonally positioned letters. The schematic representation of the binding form of these mutations and the target mRNA are also illustrated. The graph represents that introduction of A or U at the 5′ end enhanced the RNAi activity at site A of the firefly luciferase, whereas G or C did not. “MN” means non-mutated control and “NC” negative control. FIGS. 3B and 3C are for the introduction of A or U at the 5′ end that enhanced the RNAi activity at the firefly luciferase at site B. The graph B shows that the replacements of G by A and G by U result in higher interfering activity than with the non-mutated control. The same phenomenon is observed in experiments with synthesized siRNAs. As shown in FIGS. 3D and 3E, this activation phenomenon is confirmed at site C of Renilla luciferase gene, with both siRNA expression vector and synthesized siRNA.

FIG. 4 generally shows the results of Northern analysis of siRNAs in cells. FIG. 4A: HeLa cells were co-transfected with Renilla and firefly luciferases, and the non-mutated siRNA expression vector (targeted site B), ins 19 (5′ end G to A mutant), or empty vector, 24 hours later, all of the RNAs were extracted and analyzed by Northern analysis. FIG. 4B: HeLa cells were co-transfected with Renilla and firefly luciferases, and each of siRNA (10 nM). The Northern analysis was performed under the same condition as for FIG. 4A

FIG. 5 shows the results of in vitro siRNA (site B; targeted firefly luciferase) degradation assay. The non-mutated siRNA (targeted against site B of firefly luciferase) or mutant siRNA (5′ end G-to-A mutant) was incubated in HeLa S3 extract for the indicated time. The siRNAs were extracted and fractionated by polyacrylamide gel electrophoresis as shown in FIG. 5A. FIG. 5B is a graph showing the quantification of non-mutated siRNA and mutant siRNA as percent of each siRNA at 0 min.

FIG. 6 shows the effects of various types of siRNA expression vectors. The U6stem siRNA vector has the sense RNA, the loop and the antisense RNA in this order in the direction from the 5′ end to the 3′ end and the S<>AS siRNA vector has the antisense RNA, the loop and the antisense RNA in this order.

FIG. 7 shows luciferase activity with tandem and hairpin vectors.

### THE SPECIFICITY OF TRANSVERSE MUTATIONS IN siRNA

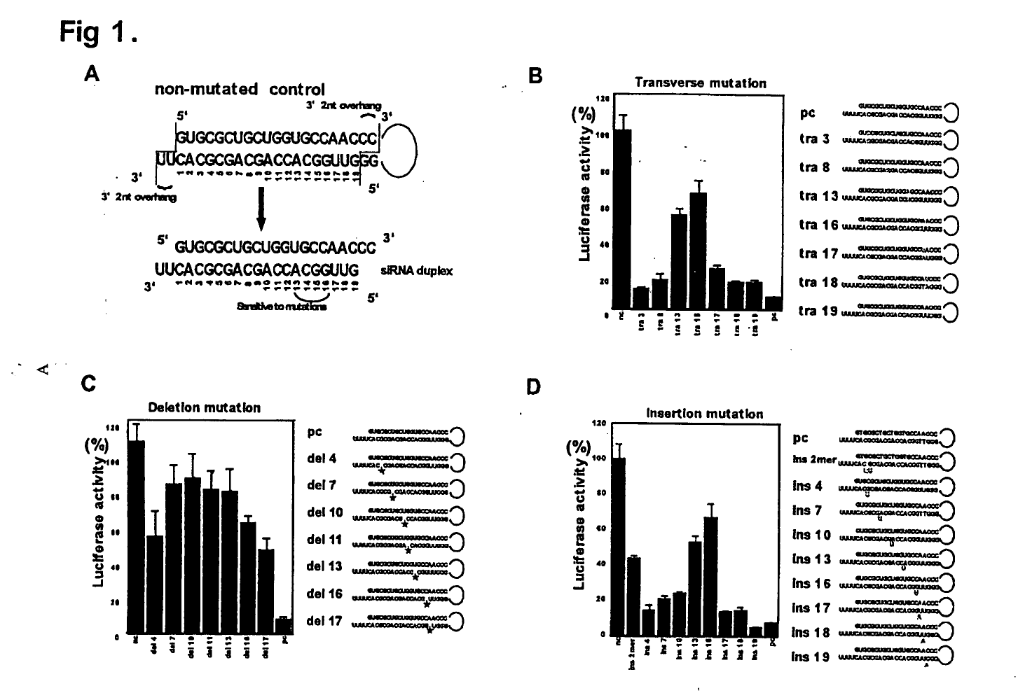

To elucidate the type and positional effects of mutations on siRNA specificity, we first constructed a series of siRNA-expression vectors with a transverse mutation, by interchanging one base pair, for example G/C to C/G or A/U to U/A. The position of mutation is numbered 1 to 19 counting from the 3′ end of the antisense strand except 2 nt 3′ overhang (FIG. 1A). Since the Dicer is reported to preferentially recognize the 5′ end of sense strand of siRNA duplexes, and cleaves them at 21 st nucleotide with 2 nt overhanging at the 3′ end (Myers et al., 2003), the final processed products should have the sequences represented by the “siRNA duplex” as shown in FIG. 1A. To confirm this structure, we carried out more than 100 comparative analyses of RNAi activity between synthesized siRNA and the corresponding siRNA expression vector at an interval of one base pair and, indeed, the results were pertinent to the structure shown in FIG. 1A. Thus, the 19th nucleotide except the 2 nt 3′ overhanging should be the 5′ end of the antisense strand after cleavage by the Dicer.

We selected firefly luciferase gene as a target of these mutated siRNA-expression vectors. HeLa S3 cells were co-transfected with the siRNA-expression vector, a firefly luciferase-expression vector, and a Renilla luciferase-expression vector. Twenty-four hours after transfection, we measured the luciferase activity by the Dual Luciferase Assay. As shown in FIG. 1B, the tra13 (transverse mutations at the 13th position) and tra16 mutations preserved little of the RNAi activity, whereas the rest of other mutants (tra3, tra8, tra17, tra18, and tra19) maintained nearly 80% interfering activity. It is very interesting that the tra17 mutant maintained high interfering activity compared with tra16, though being only one base pair away. These results point out that the transversion mutations could be permissive at or near either of the 3′ and 5′ ends, but not at the 13th to 16th positions of the antisense strand.

The Specificity of Deletion Mutations in siRNA

We also constructed deletion siRNA-expression vectors, which targeted the same sites as the transversion mutations. In each vector, we deleted one base from the antisense strand of the siRNA duplexes. The deleted bases are shown in FIG. 1C (star marks). We numbered them in the same manner as transversion mutations, del1 to del19 from 3′ end of antisense strand. Unpredictably, the deletion mutations did not maintain the same patterns of RNAi as the transversion. As shown in FIG. 1C, del14, del16 and del17 left 40% activity. However, from del7 to del13, the RNAi activity was exceedingly low, 10 to 15%. These results suggest that the siRNA has low tolerance to deletion mutations.

The Specificity of Insertion Mutations in siRNA

In the case of insertion mutations, we made a series of mutated siRNAs by inserting one base to the antisense strand. These were numbered by the same method as the transversions and deletions. When insertion is noted to occur at position N (insN), a base is actually inserted between positions N and N+1 with respect to the from −3′- to −5′ direction of the antisense RNA or the right hand side of the base at position N. As shown in FIG. 1D, ins4, ins7 and ins10 maintained the RNAi activity at a high level, quantifiably 75% to 85%. The 5′ end ins17 to ins19 also showed high interfering activity, above 80%. However, ins13 and ins16 showed only about 30 to 40% interfering activity. We also constructed a two-base pair insertion mutation at the 4 th position from 3′ end of antisense strand. The two-base mutation (ins 2 mer) maintained 40% interfering activity (FIG. 1D). This revealed that even two-base mutations could be tolerated at the 3′ side of the antisense strand. Taken all together, these results indicate that for insertion mutations more critical positions of the siRNA duplex are 5′ sides, from position 13 to position 16, than the central positions. These sensitive positions coincide with those of transversion mutants, as clearly shown in FIG. 1B.

Significantly Enhanced Stability of the Antisense Strand in Cells of Short Hairpin RNA

Generally, the interfering process is thought to involve the antisense strand of the siRNA duplex, which combines with the mRNA and directs cleavage. In the case of synthesized siRNAs, both strands, sense and antisense, potentially possess interfering activity. When we constructed hairpin type expression vectors, we experienced asymmetrical effects. To determine whether or not the sense strand of the siRNA expression vector could have interfering activity, we constructed two different types (hairpin and tandem types) of siRNA expression vectors at the same site of Renilla luciferase gene. The hairpin type vector, which is also called stem-loop type vector, involves one U6 promoter and a sense strand and an antisense strand connected by a loop are located downstream from the U6 promoter. The hairpin RNA (also called short hairpin RNA or shRNA) is processed by the Dicer and forms the siRNA duplex. The tandem type involves two U6 promoters and each promoter is responsible for the transcription of respective sense and antisense strands separately. In case of the tandem type vector, the siRNA results directly from the vector through transcription and hybridization.

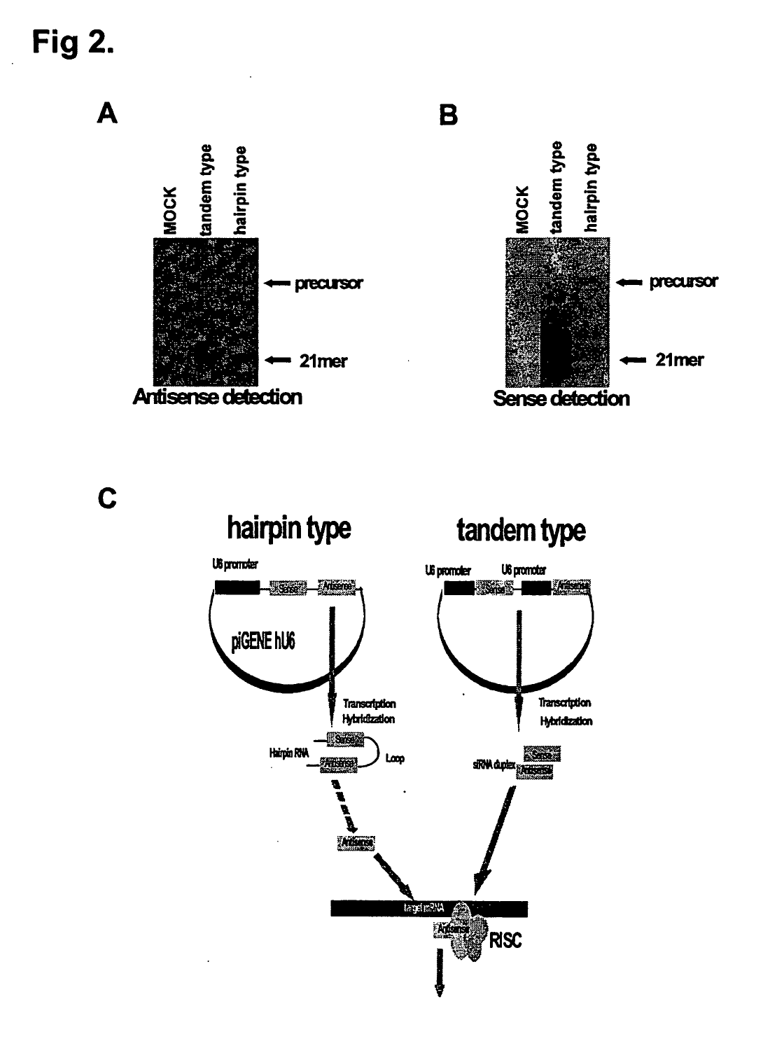

To our knowledge, no attempts have been made so far to determine the steady-state level of sense and antisense strands of siRNA derived from these two types of expression vectors. As shown in FIGS. 2A and 2B, the antisense strands of both types of expression vectors were clearly identified. However, to our surprise, the sense strand was missing from the hairpin construct even at a higher detection level (FIG. 2B), whereas the corresponding sense strand from the tandem type vector was clearly detectable, in support of the asymmetrical effect of the hairpin type vectors. These results indicate that the sense strand of the hairpin type siRNA-expression vectors would be degraded more readily and significantly higher populations of the antisense strand exist in cells. In conclusion, our analysis demonstrate for the first time that, in terms of specificity, the hairpin construct might be superior over tandem type vectors (and synthetic siRNAs) because the life time of the functionally important antisense strand is significantly longer than that of the sense strand and, thus, the unwanted RNAi effect from the sense strand can be avoided.

The Significant Enhancement of RNAi Effect by Introduction of A or U at the 5′ End of the Antisense Strand

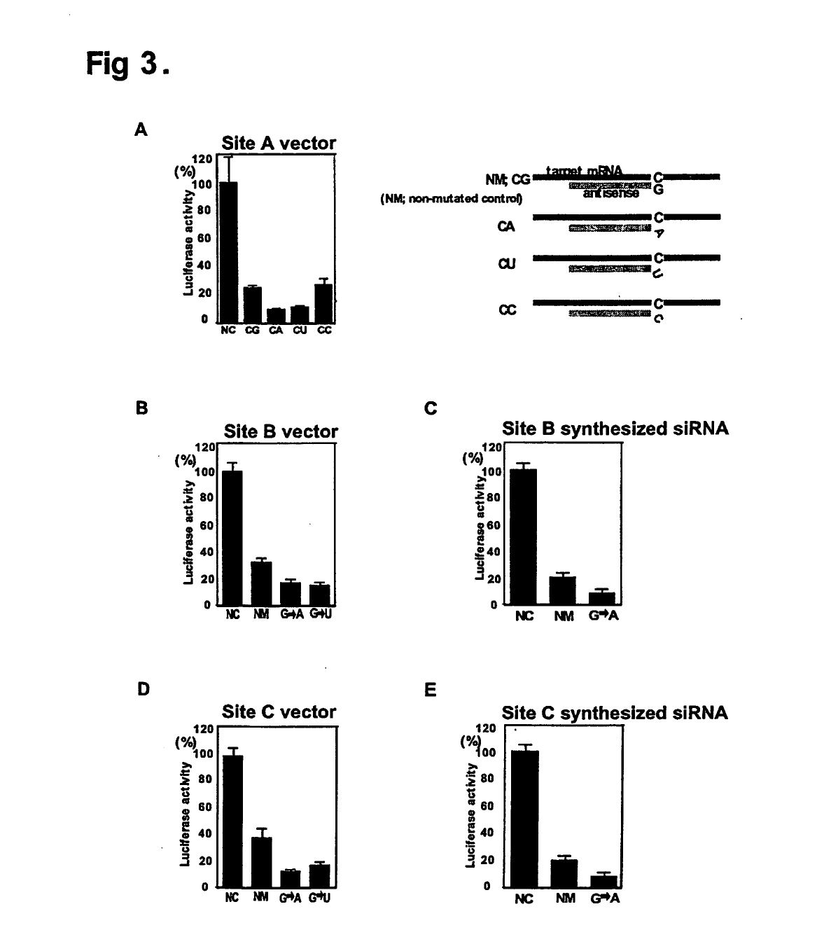

During our analysis of insertion mutations, we recognized that the 5′ end one base pair mutation, that is, 5′ end insertion mutation (ins 19, in FIG. 1C), made the siRNA activity more effective than the non-mutated control. To examine the generality of this effect, we made a series of mutated siRNA expression vectors targeting against firefly luciferase (Site A). At first, we exchanged base G found at the 5′ end to C, A or U (FIG. 3A). Among the mismatches, introduction of A or U at the 5′ end enhanced RNAi activity.

To further examine the generality of this phenomenon, we made siRNA expression vectors, targeted against other sites of firefly luciferase (Site B) and Renilla luciferase (Site C). In these vectors, we changed base G of the 5′ end to A or U. As shown in FIGS. 3B and 3D, the A and U mutations could enhance the RNAi activity. These results have confirmed the generality of the phenomenon, that base A or U at the 5′ end of the antisense strand of siRNA will make the siRNA activity higher. This activation phenomenon was also confirmed valid in the case of synthesized siRNAs (FIGS. 3C and 3E).

The Significant Enhancement of RNAi Effect of the Antisense Strand Having A or U at 5′ End Through Increased Stability in Cells

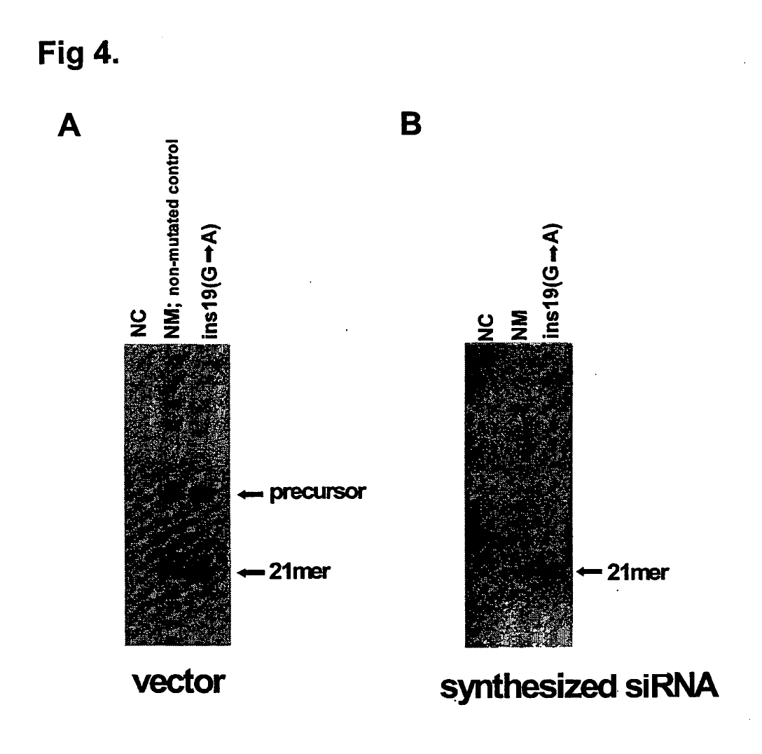

Since only the antisense strand was stabilized in cells when siRNAs were expressed from the hairpin type of construct (FIG. 2), we performed Northern analysis (FIG. 4A) to examine the effect of A or U mutation at the 5′ end on the stability in cells, because that might hint at the mechanism of the enhanced RNAi effect. We transfected siRNA expression vectors, targeted against firefly luciferase (Site B) into cells, and 24 hours later we extracted total RNAs from the cells and performed Northern analysis (FIG. 4A). Compared with the non-mutated control, vectors with the 5′end mutation, in which we exchanged G to A at the 5′ end of the antisense strand, showed a stronger signal at the 21 mer though the precursors showed nearly equal signals. This means that the antisense having A at the 5′ end could exist longer in cells than the antisense having G at the 5′ end. Therefore, the A or U of the 5′ end makes the siRNA more stable, and consequently leads to higher RNAi activity.

In order to investigate whether the stabilization effect of the 5′ end mutation is also observed in synthetic siRNA, we performed Northern analysis of synthetic siRNA. The result is shown in FIG. 4B. The 21mer band of the mutated siRNA was intense, whereas that of the non-mutated control was significantly weaker. Similar investigations were also performed against other target sites, and we obtained nearly identical results (data not shown). It should be emphasized that the observed effects did not originate from the mismatch itself by, for example, increasing the unwinding rate of the mutated duplex RNA because other mismatches such as C—C (FIG. 3A) did not enhance the RNAi effects (data not shown).

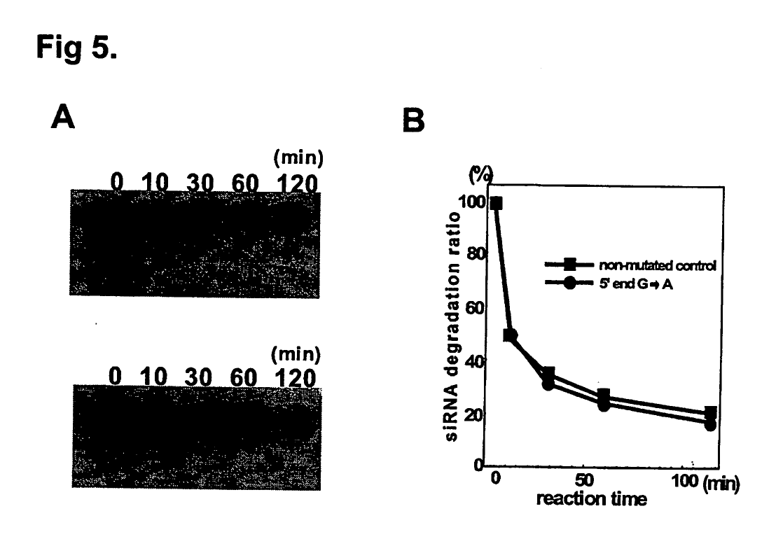

Since the A or U of the 5′ end makes the siRNA stable, and consequently leads to higher RNAi activity, we then tested whether the enhanced stability originated from resistance against general RNases or not, by performing an in vitro siRNA degradation assay. We prepared cell extracts, and added ³²P 5′-labeled siRNA After 0 minute to 2 hours incubation at 37° C., we sampled RNAs from the solution, subjected to gel electrophoresis, and measured the remaining radioactivities. By adopting this method, we could detect the level of siRNA duplex degradation in vitro. As shown in FIG. 5, the intensities of the non-mutated control with the 5′ G end were almost the same as those having a mutation of A at the 5′ end (FIG. 5A), and the degradation rates between the non-mutated control and mutation were identical (FIG. 5B). From these experiments, we conclude that the stabilization in cells of siRNA containing A at the 5′ end is not related to general RNase degradation.

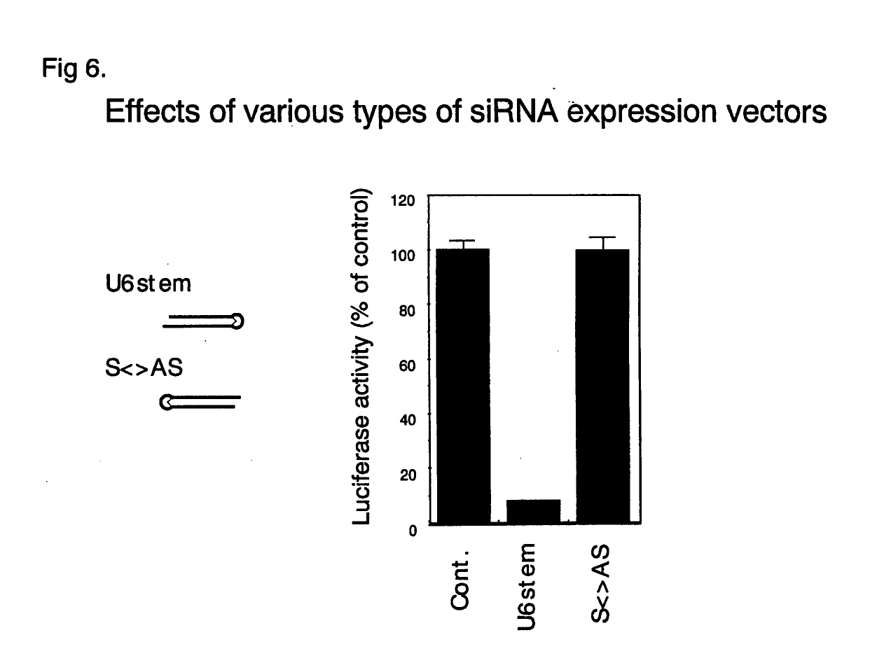

The following experiment was carried out in order to elucide effects on the inhibition of luciferase activity that the positional relationship among a sense RNA, an antisense RNA and a loop has. The three components of a vector is arranged in the order of the sense RNA, the loop and the antisense RNA in the direction from the 5′ end to the 3′ end (U6stern) in one case and in the order of the antisense RNA, the loop and the antisense RNA in the other (S<>SA) as well as a vector containing the luciferase gene were co-tranfected into cells. As a control, a vector which does not generate RNAs was used. The results are shown in FIG. 6. The U6stem vector showed much higher inhibitory effects on the luciferase expression compared with the S<>AS vector. The S<>SA siRNA vector showed very low inhibitory effect, provably because the antisense strand is degraded and only the sense strand remains.

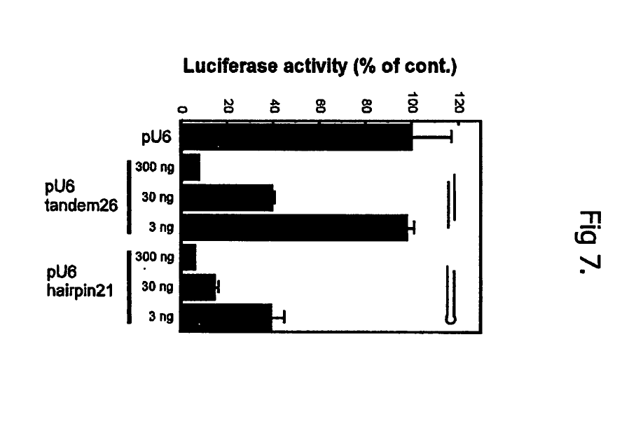

A comparison was made between a tandem type siRNA vector and a hairpin type siRNA vector. The tandem type vector (pU6tandem26) which generates a 26mer double-stranded siRNA, the hairpin type vector (pUhairpin21) which generates a 21 mer hairpin type siRNA having a loop strand between the 3′ end of the sense RNA and the 5′ end of the antisense RNA, and a vector containing a luciferase gene were co-transfected into cells. As a control, a vector (pU6) which does not generate any RNA was used. The amounts of transfection into a cell were 3, 30 or 300 ng. The results are shown in FIG. 7. The hairpin type siRNA vector showed more pronounced inhibition of luciferase activity with smaller dosages compared with the tandem type.

Remarks

Generally siRNA is thought to be highly specific in recognizing target mRNA, but recent studies (Chi et al., 2003; Jackson et al., 2003; Semizarov et al., 2003) have pointed out that this is not always the case. To summarize the past reports, there are two different opinions. One is that the RNAi effects of mutated siRNA would be extinguished (Brummelkamp et al., 2002; Elbashir et al., 2001c; Gitlin and Andino, 2003; Gitlin et al., 2002; Klahre et al., 2002). Another opinion is that one or a few base mutations would exhibit only the partial loss of activity (Amarzguioui et al., 2003; Boutla et al., 2001; Holen et al., 2003; Jacque et al., 2002). At least our present results seem to fit better with the latter opinion and the type of permitted mutation would be insertion and transverse mutations. One reason for the differences between these studies could be the assay systems, in vitro or in vivo. According to the present invention, almost all types of mutations showed at least partial loss of the activity. Elbashir and other's results might be mirroring these partial losses of activity. Thinking about the positional effects, Elbashir et al. suggested that the most specific position was the center of siRNA. On the other hand, Amarzgruinu et al. (Amarzguioui et al., 2003) suggested that siRNA generally tolerated transversion mutations and chemical modifications at the 3′end of an antisense strand, while the 5′ end exhibited low tolerance. Our present results are in agreement with the latter and make it clear the benefit of such and other mutations.

Since our analysis demonstrated that the activation depended on the 5′ end base (A or U) and not on the mismatch to the target mRNA, higher RNAi activity can always be achieved by simply changing the antisense siRNA's 5′ end base to A or U for any target sites. This should be very useful for constructing siRNAs or siRNA expression vectors. For applications such as therapeutics in which safety is very important, siRNAs should specifically knock down only the target sequence without interfering unrelated genes. The present invention related to the siRNA specificity offers safe siRNA and siRNA expression vectors. Specifically, (i) hairpin type siRNA-expression vectors might have advantage over the other systems including synthetic siRNAs because unwanted sense RNAi effect can easily be avoided as shown in FIG. 2; (ii) the antisense strand should end with A or U at the 5′ end, ensuring the in vivo stability of the transcribed siRNA and, as a result, enhancing the RNAi activity; and (iii) the adaptation of (ii) in the hairpin construct should clearly be very useful. We trust that these simple rules should help in the construction of intracellularly active siRNA vectors for basic and medical uses.

Methods

Construction of siRNA Expression Plasmids

To construct the siRNA expression plasmids, we used piGENE hU6 Vector (iGENE Therapeutic, Inc.; http://www.iGENE-therapeutics.co.jp), which contains a human U6 promoter and two Bsp MI sites. In order to compose hairpin type siRNA expression vectors, we synthesized oligonucleotides (Hokkaido System Science Co., Sapporo, Japan) with includes sense and antisense sequences, an 11-base hairpin sequence, a terminator sequence, and overhanging sequences. After annealing, the DNA fragments were ligated into the Bsp MI sites of the piGENE hU6 vector. The target gene sequences were as described (Miyagishi and Taira, 2002), and as follows (shown only the sense sequence): firefly luciferase Site A: 5′-GTG CGC TGC TGG TGC CAA C-3′; Site B: 5′-GCT ATG AAA CGA TAT GGG C-3′; Renilla luciferase Site C: 5′-GTA GCG CGG TGT ATT ATA C-3′.

Cell Culture, Transfection and Reporter Gene Assays

Hela S3 cells were cultured in Dulbecco's modified Eagle's medium (DMEM) supplemented with 1% antibiotics and 10% fetal bovine serum. Transfections were carried out using Lipofectamine™ 2000 (Invitrogen, Carlsbad, Calif.) reagents including 100 ng siRNA expression plasmid, 100 ng of firefly luciferase expression plasmid (pGL3-control vector, Promega, Madison, Wis.) and 30 ng of Renilla luciferase expression plasmid (pRL-RSV, Miyagishi et al., 2000). Twenty-four hours after transfection, firefly and Renilla luciferase activities were analyzed with Dual Luciferase Assay System (Promega, Madison, Wis.). The piGENE hU6 vector served as a negative control. To ensure equal DNA amounts, empty plasmids were added at appropriate levels in each transfection.

Northern Analysis

HeLa S3 cells were co-transfected with 1 μg siRNA expression plasmid or 10 nM siRNA targeted against firefly luciferase or siRNA targeted against Renilla luciferase, 24 h later, total RNAs were extracted from the cells with Isogen Reagent (Wako, Tokyo, Japan) according to the manufactures protocol. Total RNAs (15 μg) were size-fractionated on an 18% (wt/vol) polyacrylamide-urea gel and transferred to Hybond N+ membrane (Amersham, Little Chalfont, UK). The transferred membrane was dried at room temperature and fixed with ultraviolet light. The membrane was prehybridized in 30% formamide, 10% dextran sulfate, 5×SSC, 0.5% SDS, 1× Denhardt's solution, and 0.01 mg/ml salmon sperm DNA (Sigma Aldrich Co., Saint Louis, Mo.). Hybridizations were performed at 36° C. for 3 h with synthetic oligonucleotide probes that were complementary to the sequences of the firefly or Renilla luciferase genes as follows: firefly luciferase Site B; (sense) 5′-AAG CTA TGA AAC GATATG GG-3′, Renilla luciferase Site C; (sense) 5′-AAG TAG TGT GGT GTA TTA TA-3′, (antisense) 5′-TAT AAT ACA CCG CGC TAC TT-3′. The synthetic probes were ³²P (Amasham, Little Chalfont, UK) labeled by T4 polynucleotide kinase (Takara Shuzo Co., Kyoto, Japan). The membrane were washed with 2×SSC twice at 36° C. and analyzed with Fujix Bio-Image Analyzer BAS1000 (Fuji Photo Film Co. Ltd., Tokyo, Japan).

SiRNA Preparation

SiRNA synthesis was carried out by HSS (Hokkaido System Science, Sapporo, Japan). Ribonucleotides were annealed. The concentrations of the annealed products were confirmed by electrophoresis and subsequent ethidium bromide staining. The synthesized sequences were: firefly luciferase (Site B) non-mutated control (sense) 5′-GCU AUG AAA UGA UAU GGG CUG-3′, (antisense) 5′-GCC CAU AUC GUU UCA UAG CUU-3′; mutated (sense) 5′-GCU AUG AAA UGA UAU GGG UUG-3′, (antisense) 5′-ACC CAU AUC GUU UCA UAG CUU-3′; Renilla luciferase (Site C) non-mutated control (sense) 5′-GUA GCG CGG UGU AUU AUA CUA-3′, (antisense) 5′-GUA UAA UAC ACC GCG CUA CUU-3′; mutated (sense) 5′-GUA GCG CGG UGU AUU AUA UUA-3′, (antisense) 5′-AUA UAA UAC ACC GCG CUA CUU-3′.

In vitro siRNA Degradation Assay

HeLa S3 extracts were prepared as described bellow. Briefly, the cells were harvested and suspended in cytoplasmic buffer (10 mM HEPES pH 7.9, 10 MM KCl, 1.5 mM MgCl₂, and 0.4% NP-40) at 4° C., and stood for 10 min. The cell lysate was centrifuged for 5 minuets at 3,000 rpm, and the supernatant was recentrifugated for 60 min at 15,000 g. The protein concentration of HeLa S3 extract was 5.0 mg/ml as determined by DC protein assay (BioRad, Richmond, Calif.). The siRNA duplexes were synthesized as detailed above. The synthesized siRNA duplexes were ³²P labeled by T4 polynucleotide kinase (Takara Shuzo Co., Kyoto, Japan). The in vitro siRNA reaction buffer was prepared as described (Martinez et al., 2002). After addition of all components, the buffer, 100 nM siRNA duplex, and 50% HeLa S3 extract, we incubated the samples at 37° C. After the reaction, we extracted RNAs from the solution by phenol/chloroform/isoamil alcohol (25:24:1). The extracted RNAs were size-fractionated on an 18% polyacrylamid-urea gel. After the fixation with 10% methanol and 10% acetic acid for 45 min, the gel was analyzed with Fujix Bio-Image Analyzer BAS1000.

### References

Amarzguioui, M., Holen, T., Babaie, E. and Prydz, H. (2003) Tolerance for mutations and chemical modifications in a siRNA. Nucleic Acids Res, 31, 589-595.

Bernstein, E., Caudy, A. A., Hammond, S. M. and Hannon, G. J. (2001) Role for a bidentate ribonuclease in the initiation step of RNA interference. Nature, 409, 363-366.

Boutla, A., Delidakis, C., Livadaras, L., Tsagris, M. and Tabler, M. (2001) Short 5′-phosphorylated double-stranded RNAs induce RNA interference in Drosophila, Curr Biol, 11, 1776-1780.

Brummelkamp, T. R., Bernards, R. and Agami, R. (2002) A system for stable expression of short interfering RNAs in mammalian cells, Science, 296, 550-553.

Caplen, N. J., Parrish, S., Imani, F., Fire, A. and Morgan, R. A. (2001) Specific inhibition of gene expression by small double-stranded RNAs in invertebrate and vertebrate systems, Proc Natl Acad Sci USA, 98, 9742-9747.

Chi, J. T., Chang, H. Y., Wang, N. N., Chang, D. S., Dunphy, N. and Brown, P. O. (2003) Genomewide view of gene silencing by small interfering, RNAs. Proc Natl Acad Sci USA, 100, 6343-6346.

Dignam, J. D., Lebovitz, R. M. and Roeder, R. G. (1983) Accurate transcription initiation by RNA polymerase II in a soluble extract form isolated mammalian nuclei, Nucleic Acids Res, 11, 1475-1489.

Elbashir, S. M., Harborth, J., Lendeckel, W., Yalcin, A., Weber, K. and Tuschl, T. (2001a) Duplexes of 21-nucleotide RNAs mediate RNA interference in cultured mammalian cells, Nature, 411, 494-498.

Elbashir, S. M., Lendeckel, W. and Tuschl, T. (2001b) RNA interference is mediated by 21- and 22-nucleotide RNAs, Genes Dev, 15, 188-200.

Elbashir, S. M., Martinez, J., Patkaniowska, A., Lendeckel, W. and Tuschl, T. (2001c) Functional anatomy of siRNAs for mediating efficient RNAi in Drosophila melanogaster embryo lysate, Embo J, 20, 6877-6888.

Fire, A., Xu, S., Montgomery, M. K., Kostas, S. A., Driver, S. E. and Mello, C. C. (1998) Potent and specific genetic interference by double-stranded RNA in Caenorhabditis elegans, Nature, 391, 806-811.

Gitlin, L. and Andino, R. (2003) Nucleic acid-based immune system: the antiviral potential of mammalian RNA silencing, J Virol, 77, 7159-7165.

Gitlin, L., Karelsky, S. and Andino, R. (2002) Short interfering RNA confers intracellular antiviral immunity in human cells, Nature, 418,430-434.

Hammond, S. M., Boettcher, S., Caudy, A. A., Kobayashi, R. and Hannon, G. J. (2001) Argonaute2, a link between genetic and biochemical analyses of RNAi, Science, 293, 1146-1150.

Holen, T., Amarzguioui, M., Babaie, E. and Prydz, H. (2003) Similar behaviour of singlestrand and double-strand siRNAs suggests they act through a common RNAi pathway, Nucleic Acids Res, 31, 2401-2407.

Jackson, A. L., Bartz, S. R., Schelter, J., Kobayashi S. V., Burchard, J., Mao, M., Li, B., Cavet, G. and Linsley, P. S. (2003) Expression profiling reveals off-target gene regulation by RNAi, Nat Biotechnol, 21, 635-637.

Jacque, J. M., Triques, K. and Stevenson, M. (2002) Modulation of HIV-1 replication by RNA interference, Nature, 418, 435-438.

Klahre, U., Crete, P., Leuenberger, S. A., Iglesias, V. A. and Meins, F., Jr. (2002) High molecular weight RNAs and small interfering RNAs induce systemic posttranscriptional gene silencing in plants, Proc Natl Acad Sci USA, 99, 11981-11986.

Lee, N. S., Dohjima, T., Bauer, G., Li, H., Li, M. J., Ehsani, A., Salvaterra, P. and Rossi, J (2002) Expression of small interfering RNAs targeted against HIV-1 rev transcripts in human cells, Nat Biotechnol, 20, 500-505.

Martinez, J., Patkaniowska, A., Urlaub, H., Luhrmann, R. and Tuschl, T. (2002) Singlestranded antisense siRNAs guide target RNA cleavage in RNAi, Cell, 110, 563-574.

McCaffrey, A. P., Nakai H., Pandey, K., Huang, Z., Salazar, F. H., Xu, H., Wieland, S. F., Marion, P. L. and Kay, M. A. (2003) Inhibition of hepatitis B virus in mice by RNA interference, Nat Biotechnol, 21, 639-644.

Miyagishi, M., Fujii, R., Hatta, M., Yoshida, E., Araya, N., Nagalfuchi, A., Ishihara, S., Nakajima, T. and Fukamizu, A. (2000) Regulation of Lef-mediated transcription and p53-dependent pathway by associating beta-catenin with CBP/p300, J Biol Chem, 275, 35170-35175.

Miyagishi, M. and Taira, K. (2002) U6 promoter-driven siRNAs with four uridine 3′ overhangs efficiently suppress targeted gene expression in mammalian cells, Nat Biotechnol, 20, 497-500.

Myers, J. W., Jones, J. T., Meyer, T. and Ferrell, J. E. (2003) Recombinant Dicer efficiently converts large dsRNAs into siRNAs suitable for gene silencing, Nat Biotechnol, 21, 324-328.

Novina, C. D., Murray, M. F., Dykxhoorn, D. M., Beresford, P. J., Riess, J., Lee, S. K., Collman, R. G., Lieberman, J., Shankar, P. and Sharp, P. A. (2002) siRNA-directed inhibition of HIV-1 infection, Nat Med, 8, 681-686.

Paddison, P. J., Caudy, A. A. and Hannon, G. J. (2002) Stable suppression of gene expression by RNAi in mammalian cells, Proc Natl Acad Sci USA, 99, 1443-1448.

Paul, C. P., Good, P. D., Winer, I. and Engelke, D. R. (2002) Effective expression of small interfering RNA in human cells, Nat Biotechnol, 20, 505-508.

Semizarov, D., Frost, L., Sarthy, A., Kroeger, P., Halbert, D. N. and Fesik, S. W. (2003) Specificity of short interfering RNA determined through gene expression signatures, Proc Natl Acad Sci USA, 100, 6347-6352.

Song, E., Lee, S. K., Dykxhoorn, D. M., Novina, C., Zhang, D., Crawford, K., Cerny, J., Sharp, P. A., Lieberman, J., Manjunath, N. and Shankar, P. (2003) Sustained small interfering RNA-mediated human immunodeficiency virus type 1 inhibition in primary macrophages, J Virol, 77, 7174-7181.

Sui G., Soohoo, C., Affar el, B., Gay, F., Shi, Y. and Forrester, W. C. (2002) A DNA vector-based RNAi technology to suppress gene expression in mammalian cells. Proc Natl Acad Sci USA, 99, 5515-5520.

Yu, J. Y., DeRuiter, S. L. and Turner, D. L. (2002) RNA interference by expression of shortinterfering RNAs and hairpin RNAs in mammalian cells. Proc Natl Acad Sci USA, 99, 6047-6052.

Khvorova, A., Reynolds, A., and Jayasena, S. D., (2003) Functional siRNAs and miRNAs Exhibit Strand Bias, Cell, Vol. 115, 209-216.

Schwarz, D. S., Hutvagner, G., Du, T., Xu, Z., Aronin, N. and Zamore, P. D., (2003) Asymmetry in the Assembly of the RNAi Enzyme Complex, Cell, Vol. 115, 199-208.

1. A double-stranded RNA comprising an antisense RNA and a sense RNA, wherein the double stranded RNA has at least one substitution or insertion mutation introduced at a first, second or third nucleotide position from the 5′ end of the antisense RNA and the antisense RNA is complementary to a region of an mRNA of a target gene except the at least one substitution or insertion mutation.

2. A double-stranded RNA comprising an antisense RNA and a sense RNA, wherein the double stranded RNA has a length of 19 nt and at least one substitution or insertion mutation introduced at any of nucleotide positions 17 to 19 as counted from the 3′ end of the antisense sequence excluding an overhang and the antisense RNA is complementary to a region of an mRNA of a target gene except the at least one substitution or insertion mutation.

3. The double-stranded RNA according to claim 1, wherein the at least one substitution or insertion mutation is at least one transverse mutation.

4. The double-stranded RNA according to claim 1, which has at least one substitution or insertion mutation at the 5′ end of the antisense RNA.

5. The double-stranded RNA according to claim 1, wherein any of the at least one substitution or insertion mutation involves one nucleotide pair or one nucleotide.

6. The double-stranded RNA according to claim 1, wherein any of the at least one substitution or insertion mutation involves two nucleotide pairs or two nucleotides.

7. The double-stranded RNA according to claim 1, wherein the length of the double-stranded RNA is 15-100 nt.

8. The double-stranded RNA according to claim 1, wherein the length of the double-stranded RNA is 17-50 nt.

9. The double-stranded RNA according to claim 1, wherein the length of the double-stranded RNA is 19-30 nt.

10. The double-stranded RNA according to claim 1, further comprising an overhang having a length of 2-4 nt at the 3′ end of the antisense RNA strand.

11. The double-stranded RNA according to claim 1, wherein the double-stranded RNA is transcribed using Pol III or Pol II promoter.

12. A double-stranded RNA comprising an antisense RNA whose 5′ end nucleotide is adenosine or uridine in place of guanosine complementary to cytidine present in a region of an mRNA of a target gene, wherein the antisense RNA is complementary to the region of the mRNA of the target gene except at the 5′ end, and a sense RNA which is complementary to the region of the mRNA of the target gene.

13. A double-stranded RNA according to claim 12, wherein cytidine in the sense RNA corresponding to the replaced adenosine or uridine is replaced by uridine or adenosine respectively so as to form a complementary pair at the 5′ end of the antisense RNA.

14. A double-stranded RNA comprising an antisense RNA whose 5′ end nucleotide is adenosine or uridine in place of cytidine complementary to guanosine present in a region of an mRNA of a target gene, wherein the antisense RNA is complementary to the region of the mRNA of the target gene except at the 5′ end, and a sense RNA which is complementary to the region of the mRNA of the target gene.

15. A double-stranded RNA according to claim 14, wherein guanosine in the sense RNA corresponding to the replaced adenosine or uridine is replaced by uridine or adenosine respectively so as to form a complementary pair at the 5′ end of the antisense RNA.

16. The double-stranded RNA according to claim 12, in which the replacement of guanosine by adenosine or uridine is effected by insertion mutation.

17. The double-stranded RNA according to claim 12, wherein the length of the double-stranded RNA is 15-100 nt.

18. The double-stranded RNA according to claim 12, wherein the length of the double-stranded RNA is 17-50 nt.

19. The double-stranded RNA according to claim 12, wherein the length of the double-stranded RNA is 19-30 nt.

20. The double-stranded RNA according to claim 12, further comprising an overhang having a length of 2-4 nt at the 3′ end of the antisense strand.

21. The double-stranded RNA according to claim 12, wherein the double-stranded RNA is transcribed using Pol III or Pol II promoter.

22. A hairpin type double-stranded RNA comprising:
an antisense RNA which has at least one substitution or insertion mutation at a first, second or third nucleotide position from the 5′ end of the antisense RNA and wherein said antisense RNA is complementary to a region of an mRNA of a target gene except the at least one substitution or insertion mutation;
a sense RNA having a sequence complementary to the region of the mRNA of the target gene, and
a loop strand which connects the antisense and sense RNAs.

23. The hairpin type double-stranded RNA according to claim 22, wherein the loop strand is located between the 5′ end of the antisense RNA and the 3′ end of the sense RNA.

24. The hairpin type double-stranded RNA according to claim 22, wherein the at least one substitution mutation is at least one transverse mutation.

25. The hairpin type double-stranded RNA according to claim 22, wherein the at least one substitution mutation is located at the 5′ end of the antisense RNA.

26. The hairpin type double-stranded RNA according to claim 22, wherein the double-stranded RNA has a length of 17-30 nt excluding the loop strand.

27. The hairpin type double-stranded RNA according to claim 22, wherein the double-stranded RNA has a length of 19-21 nt excluding the loop strand.

28. The hairpin type double-stranded RNA according to claim 22, wherein the double-stranded RNA has a length of 19 nt excluding the loop strand.

29. The hairpin type double-stranded RNA according to claim 22, wherein the double-stranded RNA has a length of 21 nt excluding the loop strand and the at least one substitution mutation is at positions 16-21 as counted from the 3′ end of the antisense sequence excluding any possible overhang.

30. A hairpin type double-stranded RNA comprising:
an antisense RNA whose 5′ end nucleotide is adenosine or uridine in place of guanosine complementary to cytidine present in a region of an mRNA of a target gene, wherein the antisense RNA is complementary to the region of the mRNA of the target gene except at the 5′ end,
a sense RNA which is complementary to the region of the mRNA of the target gene, and
a loop strand that connects the antisense and sense RNAs.

31. The hairpin type double-stranded RNA according to claim 30, wherein the loop strand is located between the 5′ end of the antisense RNA and the 3′ end of the sense RNA.

32. The hairpin type double-stranded RNA according to claim 30, wherein cytidine in the sense RNA corresponding to the replaced adenosine or uridine is replaced by uridine or adenosine respectively so as to form a complementary pair at the 5′ end of the antisense RNA.

33. A hairpin type double-stranded RNA comprising:
an antisense RNA whose 5′ end nucleotide is adenosine or uridine in place of cytidine complementary to guanosine present in a region of an mRNA of a target gene, wherein the antisense RNA is complementary to the region of the mRNA of the target gene except at the 5′ end,
a sense RNA which is complementary to the region of the mRNA of the target gene a loop strand that connects the antisense and sense RNAs, and
a loop strand that connects the antisense and sense RNAs.

34. The hairpin type double-stranded RNA according to claim 33, wherein tie loop strand is located between the 5′ end of the antisense RNA and the 3′ end of the sense RNA.

35. The hairpin type double-stranded RNA according to claim 33, wherein guanosine in the sense RNA corresponding to the replaced adenosine or uridine is replaced by uridine or adenosine respectively so as to form a complementary pair at the 5′ end of the antisense RNA.

36. The hairpin type double-stranded RNA according to claim 35, in which the replacement of guanosine by adenosine or uridine is generated by insertion mutation.

37. An antisense RNA which has at least one substitution or insertion mutation near the 5′ end of the antisense RNA and wherein said antisense RNA is complementary to a region of an mRNA of a target gene except the at least one substitution or insertion mutation.

38. The antisense RNA according to claim 37, wherein the at least one substitution or insertion mutation is located at the 5′ end of the antisense RNA.

39. The antisense RNA according to claim 37, wherein the antisense RNA has a length of 19 nt and the at least one substitution or insertion mutation is at any of nucleotide positions 17 to 19 as counted from the 3′ end of the antisense sequence excluding an overhang.

40. The antisense RNA according to claim 37, wherein the antisense RNA has a length of 21 nt and the at least one substitution or insertion mutation is at any of nucleotide positions 19 to 21 as counted from the 3′ end of the antisense sequence excluding an overhang.

41. The antisense RNA according to claim 37, wherein the antisense RNA has a length of 15-100 nt excluding any possible overhang.

42. The antisense RNA according to claim 37, wherein the antisense RNA has a length of 17-50 nt excluding any possible overhang.

43. The antisense RNA according to claim 37, wherein the antisense RNA has a length of 19-30 nt excluding any possible overhang.

44. The antisense RNA according to claim 37, wherein the antisense RNA has a length of 19 nt excluding any possible overhang.

45. The antisense RNA according to claim 37, further comprising an overhang having a length of 2-4 nt at 3′ end.

46. A hairpin type vector for inhibiting expression of an mRNA of a target gene, comprising:
an antisense DNA strand coding for an antisense RNA which has at least one mutation that corresponds to a substitution or insertion mutation at a first, second or third nucleotide position from the 5′ end of a double-stranded RNA resulting therefrom, wherein the antisense RNA is complementary to a region of an mRNA of a target gene except the at least one substitution or insertion mutation;
a corresponding sense DNA strand;
a DNA strand cording for a loop strand; and
a promoter operatively linked to said DNA strands.

47. The hairpin type vector according to claims 46, wherein the DNA strand cording for the loop strand is located between the 5′ end of the antisense DNA and the 3′ end of the sense DNA.

48. The hairpin type vector according to claim 46, wherein the at least one substitution or insertion mutation is at least one transverse mutation.

49. The hairpin type vector according to claim 46, further comprising a terminator which is located downstream of the antisense DNA and capable of terminating the transcription of the antisense DNA.

50. The hairpin type vector according to claim 46, wherein guanosine at the 5′ end of the antisense RNA is replaced by adenosine or uridine.

51. The hairpin type vector according to claim 50, wherein the replacement is effected by insertion mutation.

52. A tandem type vector for inhibiting expression of an mRNA of a target gene, comprising:
an antisense DNA strand coding for an antisense RNA which has at least one mutation that corresponds to a substitution or insertion mutation at a first, second or third nucleotide position from a 5′ end of a double-stranded RNA resulting therefrom and is otherwise complementary to the mRNA;
a promoter operatively liked to the antisense DNA strand;
a corresponding sense DNA strand;
a promoter operatively linked to the sense DNA strand.

53. The tandem type vector according to claim 52, wherein the at least one substitution or insertion mutation is at least one transverse mutation.

54. The tandem type vector according to claim 52, further comprising a terminator which is located downstream of the antisense DNA and capable of terminating the transcription of the antisense DNA.

55. The tandem type vector according to claim 52, wherein guanosine at the 5′ end of the antisense RNA is replaced by adenosine or uridine.

56. The tandem type vector according to claim 52, wherein the replacement is effected by insertion mutation.

57. A method for inhibiting expression of an mRNA of a target gene in a cell, comprising the steps of:
preparing a double-stranded RNA comprising an antisense RNA which has at least one substitution or insertion mutation at a first, second or third nucleotide position from the 5′ end of the antisense RNA and wherein said antisense RNA is complementary to some region of an mRNA of a target gene except the at least one substitution or insertion mutation; and a sense RNA having a sense sequence complementary to the antisense RNA;
introducing the double-stranded RNA into a cell which contains the mRNA; and
selecting the antisense RNA.

58. The method according to claim 57, wherein the double-stranded RNA further comprises a loop strand between the 5′ end of the antisense RNA and the 3′ end of the sense RNA.

59. A method for inhibiting expression of an mRNA of a target gene in a cell, comprising the steps of:
preparing a vector for inhibiting expression of an mRNA of a target gene, the vector comprising an antisense DNA strand coding for an antisense RNA having an antisense sequence whose 5′ end is adenosine or uridine, wherein the antisense sequence other than the 5′ end nucleotide of the antisense sequence is complementary to a region of the mRNA, and a promoter operatively linked to the antisense DNA;
introducing the vector into a cell which contains the target gene; and
selecting a cell that expresses the antisense RNA.

60. A cell which has been transformed using the double-stranded RNA according to claim 1.

61. A cell which has been transformed using the hairpin type double-stranded RNA according to claim 22.

62. A cell which has been transformed using the antisense RNA according to claim 37.

63. A cell which has been transformed using the hairpin type RNA vector according to claim 46.

64. A non-human animal which has cells that have been transformed using the double-stranded RNA according to claim 1.

65. A non-human animal which has cells that have been transformed using the hairpin type double-stranded RNA according to claim 22.

66. A non-human animal which has cells that have been transformed using the antisense RNA according to claim 37.

67. A non-human animal which has cells that have been transformed using the vector according to claim 46.

68. A plant which has cells that have been transformed using the double-stranded RNA according to claim 1.

69. A plant which has cells that have been transformed using the hairpin type double-stranded RNA according to claim 22.

70. A plant which has cells that have been transformed using the antisense RNA according to claim 37.

71. A plant which has cells that have been transformed using the vector according to claim 46.

72. A composition containing a vehicle and the double-stranded RNA according to claim 1.

73. A composition containing a vehicle and the hairpin type RNA according to claim 22.

74. A composition containing a vehicle and the antisense RNA according to claim 37.

75. A composition containing a vehicle and the vector according to claim 46.

76. A method for enhancing an effect of siRNA wherein a loop strand is located between the 5′ end of the antisense RNA and the 3′ end of the sense RNA.


SEQUENCE LISTING (50 sequences)

SEQ ID NO: 1 - DNA, 19 bp, Artificial Sequence
  Description of Artificial Sequence Synthetic oligonucleotide
gtgcgctgctggtgccaac

SEQ ID NO: 2 - DNA, 19 bp, Artificial Sequence
  Description of Artificial Sequence Synthetic oligonucleotide
gctatgaaacgatatgggc

SEQ ID NO: 3 - DNA, 19 bp, Artificial Sequence
  Description of Artificial Sequence Synthetic oligonucleotide
gtagcgcggtgtattatac

SEQ ID NO: 4 - DNA, 20 bp, Artificial Sequence
  Description of Artificial Sequence Synthetic oligonucleotide
aagctatgaaacgatatggg

SEQ ID NO: 5 - DNA, 20 bp, Artificial Sequence
  Description of Artificial Sequence Synthetic oligonucleotide
aagtagtgtggtgtattata

SEQ ID NO: 6 - DNA, 20 bp, Artificial Sequence
  Description of Artificial Sequence Synthetic oligonucleotide
tataatacaccgcgctactt

SEQ ID NO: 7 - RNA, 21 nt, Artificial Sequence
  Description of Artificial Sequence Synthetic oligonucleotide
gcuaugaaaugauaugggcug

SEQ ID NO: 8 - RNA, 21 nt, Artificial Sequence
  Description of Artificial Sequence Synthetic oligonucleotide
gcccauaucguuucauagcuu

SEQ ID NO: 9 - RNA, 21 nt, Artificial Sequence
  Description of Artificial Sequence Synthetic oligonucleotide
gcuaugaaaugauauggguug

SEQ ID NO: 10 - RNA, 21 nt, Artificial Sequence
  Description of Artificial Sequence Synthetic oligonucleotide
acccauaucguuucauagcuu

SEQ ID NO: 11 - RNA, 21 nt, Artificial Sequence
  Description of Artificial Sequence Synthetic oligonucleotide
guagcgcgguguauuauacua

SEQ ID NO: 12 - RNA, 21 nt, Artificial Sequence
  Description of Artificial Sequence Synthetic oligonucleotide
guauaauacaccgcgcuacuu

SEQ ID NO: 13 - RNA, 21 nt, Artificial Sequence
  Description of Artificial Sequence Synthetic oligonucleotide
guagcgcgguguauuauauua

SEQ ID NO: 14 - RNA, 21 nt, Artificial Sequence
  Description of Artificial Sequence Synthetic oligonucleotide
auauaauacaccgcgcuacuu

SEQ ID NO: 15 - RNA, 21 nt, Artificial Sequence
  Description of Artificial Sequence Synthetic oligonucleotide
gugcgcugcuggugccaaccc

SEQ ID NO: 16 - RNA, 23 nt, Artificial Sequence
  Description of Artificial Sequence Synthetic oligonucleotide
ggguuggcaccagcagcgcacuu

SEQ ID NO: 17 - RNA, 21 nt, Artificial Sequence
  Description of Artificial Sequence Synthetic oligonucleotide
guuggcaccagcagcgcacuu

SEQ ID NO: 18 - RNA, 25 nt, Artificial Sequence
  Description of Artificial Sequence Synthetic oligonucleotide
ggguuggcaccagcagcgcacuuuu

SEQ ID NO: 19 - RNA, 21 nt, Artificial Sequence
  Description of Artificial Sequence Synthetic oligonucleotide
guccgcugcuggugccaaccc

SEQ ID NO: 20 - RNA, 25 nt, Artificial Sequence
  Description of Artificial Sequence Synthetic oligonucleotide
ggguuggcaccagcagcggacuuuu

SEQ ID NO: 21 - RNA, 21 nt, Artificial Sequence
  Description of Artificial Sequence Synthetic oligonucleotide
gugcgcuccuggugccaaccc

SEQ ID NO: 22 - RNA, 25 nt, Artificial Sequence
  Description of Artificial Sequence Synthetic oligonucleotide
ggguuggcaccaggagcgcacuuuu

SEQ ID NO: 23 - RNA, 21 nt, Artificial Sequence
  Description of Artificial Sequence Synthetic oligonucleotide
gugcgcugcuggagccaaccc

SEQ ID NO: 24 - RNA, 25 nt, Artificial Sequence
  Description of Artificial Sequence Synthetic oligonucleotide
ggguuggcuccagcagcgcacuuuu

SEQ ID NO: 25 - RNA, 21 nt, Artificial Sequence
  Description of Artificial Sequence Synthetic oligonucleotide
gugcgcugcuggugcgaaccc

SEQ ID NO: 26 - RNA, 25 nt, Artificial Sequence
  Description of Artificial Sequence Synthetic oligonucleotide
ggguucgcaccagcagcgcacuuuu

SEQ ID NO: 27 - RNA, 21 nt, Artificial Sequence
  Description of Artificial Sequence Synthetic oligonucleotide
gugcgcugcuggugccuaccc

SEQ ID NO: 28 - RNA, 25 nt, Artificial Sequence
  Description of Artificial Sequence Synthetic oligonucleotide
ggguaggcaccagcagcgcacuuuu

SEQ ID NO: 29 - RNA, 21 nt, Artificial Sequence
  Description of Artificial Sequence Synthetic oligonucleotide
gugcgcugcuggugccauccc

SEQ ID NO: 30 - DNA, 25 bp, Artificial Sequence
  Description of Combined DNA/RNA Molecule Synthetic oligonucleotide
gggatggcaccagcagcgcacuuuu

SEQ ID NO: 31 - RNA, 21 nt, Artificial Sequence
  Description of Artificial Sequence Synthetic oligonucleotide
gugcgcugcuggugccaagcc

SEQ ID NO: 32 - RNA, 25 nt, Artificial Sequence
  Description of Artificial Sequence Synthetic oligonucleotide
ggcuuggcaccagcagcgcacuuuu

SEQ ID NO: 33 - RNA, 24 nt, Artificial Sequence
  Description of Artificial Sequence Synthetic oligonucleotide
ggguuggcaccagcagccacuuuu

SEQ ID NO: 34 - RNA, 24 nt, Artificial Sequence
  Description of Artificial Sequence Synthetic oligonucleotide
ggguuggcaccagcgcgcacuuuu

SEQ ID NO: 35 - RNA, 24 nt, Artificial Sequence
  Description of Artificial Sequence Synthetic oligonucleotide
ggguuggcaccgcagcgcacuuuu

SEQ ID NO: 36 - RNA, 24 nt, Artificial Sequence
  Description of Artificial Sequence Synthetic oligonucleotide
ggguuggcacagcagcgcacuuuu

SEQ ID NO: 37 - RNA, 24 nt, Artificial Sequence
  Description of Artificial Sequence Synthetic oligonucleotide
ggguuggcccagcagcgcacuuuu

SEQ ID NO: 38 - RNA, 24 nt, Artificial Sequence
  Description of Artificial Sequence Synthetic oligonucleotide
ggguugcaccagcagcgcacuuuu

SEQ ID NO: 39 - RNA, 24 nt, Artificial Sequence
  Description of Artificial Sequence Synthetic oligonucleotide
ggguggcaccagcagcgcacuuuu

SEQ ID NO: 40 - DNA, 21 bp, Artificial Sequence
  Description of Artificial Sequence Synthetic oligonucleotide
gtgcgctgctggtgccaaccc

SEQ ID NO: 41 - DNA, 25 bp, Artificial Sequence
  Description of Combined DNA/RNA Molecule Synthetic oligonucleotide
gggttggcaccagcagcgcacuuuu

SEQ ID NO: 42 - DNA, 27 bp, Artificial Sequence
  Description of Combined DNA/RNA Molecule Synthetic oligonucleotide
gggttggcaccagcagcguucacuuuu

SEQ ID NO: 43 - RNA, 26 nt, Artificial Sequence
  Description of Artificial Sequence Synthetic oligonucleotide
ggguuggcaccagcagcugcacuuuu

SEQ ID NO: 44 - DNA, 26 bp, Artificial Sequence
  Description of Combined DNA/RNA Molecule Synthetic oligonucleotide
gggttggcaccagcuagcgcacuuuu

SEQ ID NO: 45 - RNA, 26 nt, Artificial Sequence
  Description of Artificial Sequence Synthetic oligonucleotide
ggguuggcaccuagcagcgcacuuuu

SEQ ID NO: 46 - RNA, 26 nt, Artificial Sequence
  Description of Artificial Sequence Synthetic oligonucleotide
ggguuggcuaccagcagcgcacuuuu

SEQ ID NO: 47 - RNA, 26 nt, Artificial Sequence
  Description of Artificial Sequence Synthetic oligonucleotide
ggguuuggcaccagcagcgcacuuuu

SEQ ID NO: 48 - RNA, 26 nt, Artificial Sequence
  Description of Artificial Sequence Synthetic oligonucleotide
ggguauggcaccagcagcgcacuuuu

SEQ ID NO: 49 - RNA, 26 nt, Artificial Sequence
  Description of Artificial Sequence Synthetic oligonucleotide
gggauuggcaccagcagcgcacuuuu

SEQ ID NO: 50 - RNA, 26 nt, Artificial Sequence
  Description of Artificial Sequence Synthetic oligonucleotide
ggaguuggcaccagcagcgcacuuuu